(19) United States
(12) Patent Application Publication
(10) Pub. No.: US 2020/0275874 A1
(43) Pub. Date: Sep. 3, 2020

(54) Methods and Automatic System to Identify Who is Victims of Abuse Voice to Skull & Remote Neural Monitoring Technology and Identify Who is Remote Attacker or Operator Using Device of Voice to Skull & Remote Neural Monitoring

(71) Applicant: Da LI, West Covina, CA (US)

(72) Inventor: Da LI, West Covina, CA (US)

(21) Appl. No.: 16/558,040

(22) Filed: Aug. 31, 2019

Related U.S. Application Data
(60) Provisional application No. 62/812,915, filed on Mar. 1, 2019.

(51) Int. Cl. A61B 5/16; A61B 5/00

(52) U.S. Cl. CPC A61B 5/165; A61B 5/0006; A61B 5/0022; A61B 5/164

(57) ABSTRACT

A method to identify victim of abuse and remote operator using voice to skull and remote neural monitoring, comprising the steps of: using a database in a computer consisting of one or more of disgusting items, funny items, anti-political trend and anti-religious belief items, countries and races items, foreign clubs or organizations items, languages and dialects items, and scientific knowledge items, tested person selecting one item which only causes the remote operator to have psychological response and does not cause the tested person to have psychological response, instructing the tested person to launch imaginary psychological attack, assigning test questions based on the imaginary psychological attack and obtaining answers from the tested person in response to the test questions, then using polygraph meter to analysis the answers and assigning true or false status to the answers.

### CROSS REFERENCE OF RELATED APPLICATION

This application claims the benefit of U.S. Provisional Patent Application No. 62/812,915, filed Mar. 1, 2019. This application is incorporated by reference herein in their entirety.

### BACKGROUND OF THE PRESENT INVENTION

### Field of Invention

The present invention relates to a method to identify who is victims of abuse voice to skull and remote neural monitoring technology to and identify who is remote attacker or operator using device of voice to skull and remote neural monitoring.

### Description of Related Arts

Origin of the problem: from 2015 to 2018, there were large amount of victims in 22 provinces in China, Shanghai and Beijing municipalities have large number of collective protests against RNM & V2K technology abuse.

In addition, on Oct. 1, 2015, representatives from 17 countries in Berlin, Germany, hold a “Covered Harassment Conference”. The covered harassment is same thing with voice to skull & remote neural monitoring technology.

After a lot of times contacting with the victims in China mainland WeChat, emails and telephones, these victims' thoughts were read and harassed at same time by some unknown Chinese institutions abusing these technology.

According to Zhong Zhiyong, the organizer and leader of anti-V2k & RNM torture in 22 provinces and cities in mainland China (Zhong Zhiyong's affairs were also reported by the media “The Epoch Times” and he and other victims representatives also received interview with “New Tang Dynasty TV”). He told me directly by phone and WeChart almost all victims have the same experience: quickly being read thoughts and could use their thoughts to hold silent thoughts conversations with operators (ROD) using devices of V2K & RNM. Including a Chinese victim has been a lawful permanent resident in United States is still quickly being read thoughts and could use his thoughts to hold silent thoughts conversations with operators (ROD) using devices of V2K & RNM. But all these RODs are in P.R.China.

Another Chinese leader anti-abuse technology of V2K & RNM, Mr. Yao Dou-jie live in Shenzhen China who collect large information also confirmed same cases above.

Generally three to five operators (ROD) of V2k & RNM devices in group in turn concentrate one victim via satellite positioning technology to realize remote harassment, the technology is mainly based on these US technology patents and China patents which describe the technology of V2K & RNM are extracting the brain's characteristic wave frequency first (like fingerprints, each person's brain characteristic wave frequency is different), then codes the brain characteristic wave frequency by computer software technology and remotely locks the frequency and monitors the victim's brain wave frequency using computer software technology and satellite positioning technology.

The bases of these technology are microwave auditory effect or call Frey effect and neural coding technology.

In such way, three to five operators (ROD) using devices of V2K & RNM in groups perform active psychological attacks on victim's brain with some negative emotions, but the effect of this kind of psychological reactions can be two-ways or two directions between victim and operators (RODs) using devices of V2K & RNM.

In past of years, FBI's investigation on some cases related similar technology attacking.

This is not a scientific fiction, because the following US patents and China patents tell us it is true and existing technologies:

1. U.S. Pat. No. 6,470,214B1 Method and device for implementing the radio frequency hearing effect, US Air Force.

2. The US public technology patent for remote reading human thoughts is U.S. Pat. No. 3,951,134A: Apparatus and method for remotely monitoring and altering brain waves.

3. U.S. Pat. No. 6,011,991A explained how read human thoughts remotely via satellite.

4. CN 2008202247769.1 China use the tech both in peaceful time and war time.

Till now we have confirmed these technologies existing with US patents and China patents.

5. US company QU-wave.com also explains what is V2K.

Till now, there is not any method to identify both the victims and those remote operators (RODs) using devices of V2K & RNM around world. These abuse has been developed global and transnational, so the invention is the first set of method in the world which can identify both victims and remote operators (RODs) using devices of V2K & RNM accurately in high degree by means of computer technology.

### SUMMARY OF THE PRESENT INVENTION

An object of the present invention is to provide a methods to identify both the victims and those remote operators using devices of V2K & RNM around world. This is the first set invention of identification with computer and automatic technology around world which can automatically generate identification results, except checking polygraph meter with human, all other parts of the system achieves automation.

The method of the present invention:

Abbreviation:

ROD: remote operators using devices of V2K & RNM technology

ISA:an identification system administrator

TP: a tested person who originally claims him/her as a victim

Step 1. Ask a TP who was a victim to use of RODs reading his/her thoughts to launch multiple reverse psychological attacks to those RODs to cause or stimulate ROD's psychological response, the base of technology comes from US patents and China patents.

Step 2. Find out all kinds of characteristics of psychological response both in the victim's and ROD's by psychological analysis tech., the base of technology come from basic psychological analysis.

Step 3. Find the different characteristics of psychological responses between the victim's (TP's) and RODs' using relative comparing method; the base of theory come from quantitative science and statistics.

Step 4. Use of automatic control decay curve (PID control regulate curve) to prove the method of identification is accurate, the base of theory come from automatic control theory.

The present invention utilizes the victim's thoughts to be read and the victim can use his/her thoughts to establish brain thoughts talking communication with the remote operator using device (ROD) of V2K & RNM, so the invention ask the victim to use his/her thoughts intentionally choose classified content from data base of psychological reaction in a computer to attack those remote operators using device (RODs) via satellite. Because RODs are human beings and they should make psychological response on their device when they receive the victim's remote psychological attacking via satellite and then in turn the victim can feel and sense the RODs' psychological response.

According theory of psychoanalysis: the psychological response between an active psychological attacker and a psychological attacked person passively have completely different characteristics, so these different psychological responses caused by selective classified psychological attacks can be used both to identify a victim (TP) and those remote operators using device (RODs) of RNM & V2K via satellite.

From the theory of automatic control decay curve: multiple PID adjustments can achieve a correct control requirements, so the above victim (TP) who use multiple classified and deliberate psychologically attacks the remote operators using device (RODs) V2K & RNM can gradually approach the correct identifying himself/herself is a victim (TP) and identify who are remote operators using device (RODs) RNM & V2K via satellite, with the help of polygraph, the invention ensures the accuracy of identification.

Additional advantages and features of the invention will become apparent from the description which follows, and may be realized by means of the instrumentalities and combinations particular point out in the appended claims.

Still further objects and advantages will become apparent from a consideration of the ensuing description and drawings. These and other objectives, features, and advantages of the present invention will become apparent from the following detailed description, the accompanying drawings, and the appended claims.

### BRIEF DESCRIPTION OF THE DRAWINGS

FIG. 1 is a schematic diagram showing an operation of the human and machine according to a preferred embodiment of the present invention.

FIG. 2A to FIG. 2F illustrates a first set of flowcharts for identify a victim according to the present invention.

FIG. 3A to FIG. 3E illustrates a second set of flowcharts for identify all kinds status of remote operator or attacker (ROD's) according to the present invention.

FIG. 4 illustrates an identification decay curve.

FIG. 5 illustrates examples of disgusting things.

FIG. 6 illustrates examples of funny stories.

FIG. 7 illustrates examples of imaginary political attack.

FIG. 8 illustrates examples of imaginary religious attack.

FIG. 9 illustrates examples of imaginary national attack.

FIG. 10 illustrates how TP use HMI touch screen which connects with a controller and a computer to achieve automatic identification.

FIG. 11A illustrates how TP use HMI touch screen which connects with a controller and a computer to achieve automatic identification.

FIG. 11B illustrates how TP use HMI touch screen which connects with a controller and a computer to achieve automatic identification.

FIG. 12A illustrates how TP use HMI touch screen which connects with a controller and a computer to achieve automatic identification.

FIG. 12B illustrates how TP use HMI touch screen which connects with a controller and a computer to achieve automatic identification.

FIG. 13 illustrates how TP use HMI touch screen which connects with a controller and a computer to achieve automatic identification.

FIG. 14 illustrates how TP use HMI touch screen which connects with a controller and a computer to achieve automatic identification.

### DETAILED DESCRIPTION OF THE PREFERRED EMBODIMENT

V2K: What is ‘Voice to Skull’? . . . Voice to skull is the transmission of voice, or any other audible or subliminal sound, directly into the hearing sense of a victim. So the invention is based on technologies about of RNM & V2K technology around the world. The painful experience described by victims is consistent with the effect of these technologies in relation to V2K which were described in US patents and China patent.

The invention is based on these V2K & RNM device operated by human beings and these operators show themselves emotions when they are using these devises to harass victims. On the other hand, victims can feel and sense operator's emotions. This emotional interaction between victim and operators is interactive when these V2K & RNM devices are running.

The reason to confirm V2K & RNM device operated by human beings is human brain can forget something, because operators frequently forget somethings related victim's history, but victims do not forget the things in himself or herself history. On the other hand, these computer memory and robot memory with artificial intelligent never lost data when programmer programs data into their memory, so only human has memory error, but computer's CPU never has memory data read and write error. So the invention can analyze operator's human emotion which a victim can feel and sense to identify these voice to skull is not victim's auditory hallucination, but they are emotional response from those remote operators. Because there are obvious differences between victim's emotion reaction and those emotions response belong to remote operators using devices of V2K & RNM, so if using the invention method, you can find these differences clearly.

### STATEMENT OF GOVERNMENT INTEREST

The invention can help victims to confirm their grieves and get justice, on the other hand, it can help law enforcement like FBI to find out the fact of cases, as this remote technology is global and transnational crime, so if all kinds of victims enter United States in some related cases, FBI can use the invention as a tool to analysis and identify who are operators (or call them remote attacker), then these operators' (or call them remote attacker) nationality and race; religious belief and political belief, scale of language and dialect, scale of scientific knowledge, even identify these attacker or operator belong to which organization.

For example, FBI investigated the case of Washington D.C. navy yard gun case, CNN reported on September 26,2013, the gunman suffered “Low frequency attacks” and that attack drove him to kill. If similar cases happen, the invention can help FBI to investigate.

Declaration: In this invention, all political, religious attacks or attack a country are human brain thoughts imagination attacks with viewing videos, pictures & other materials in computer databases in non-public site, there is neither any verbal attack nor behavioral attack. So do not cause insulting anyone.

Description of the method of the present invention:

Abbreviation:

PM: polygraph meter (computerized digital)

ISA: an identification system administrator

TP: a tested person

RODs: remote operators using devices of V2K & RNM technology

V2K: Voice to Skull

RNM: Remote Neural Monitoring Technology

HMI: human machine interface

The invention use of victim's (TP's) intentional selective and classified thoughts to launch some psychological attacks to those remote and covered operators (RODs) using devices of V2K & RNM to stimulate those operator's (ROD's) corresponding and classified emotional responses which victim (TP) can feel and sense.

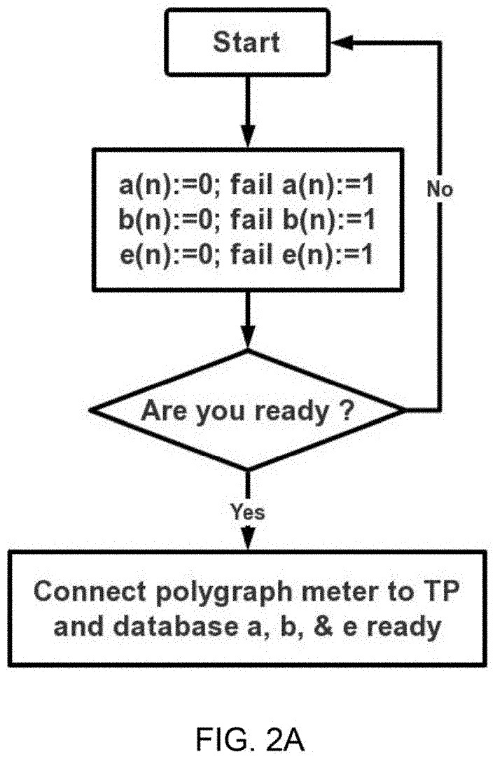

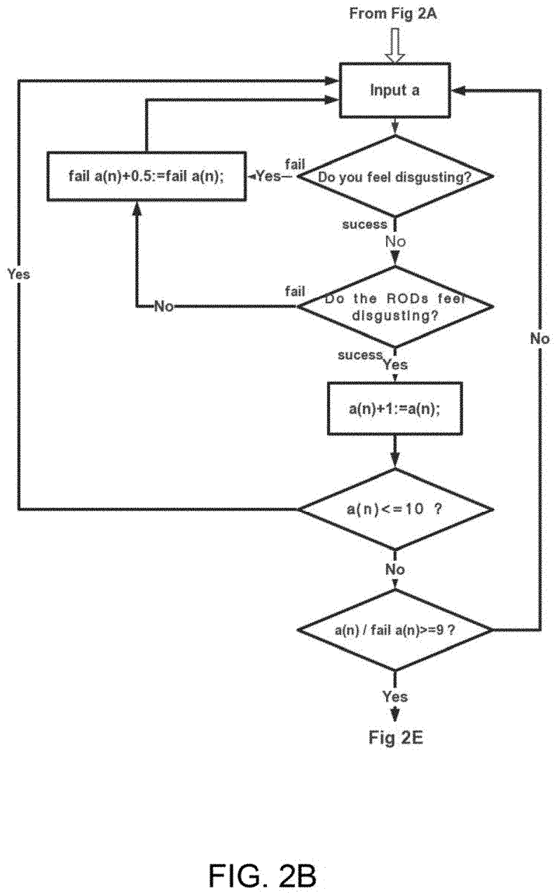

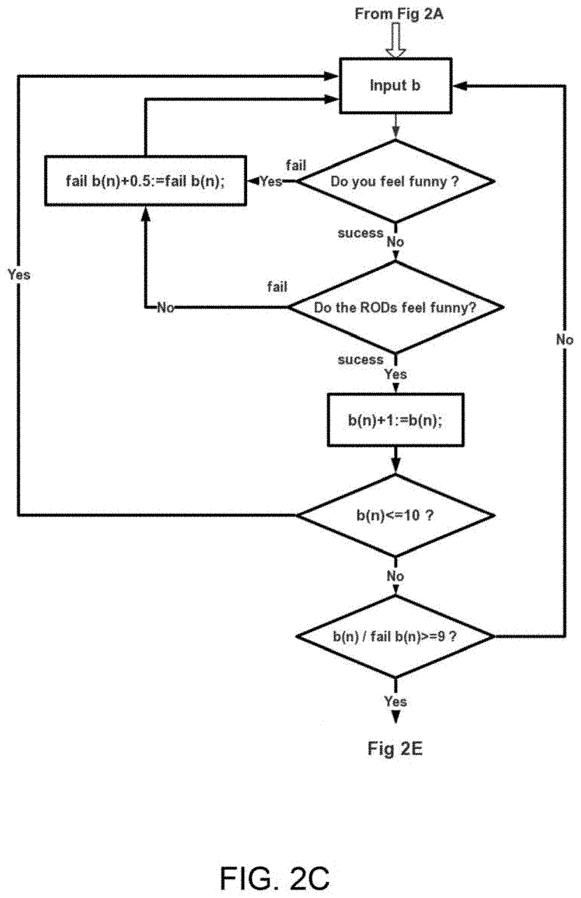

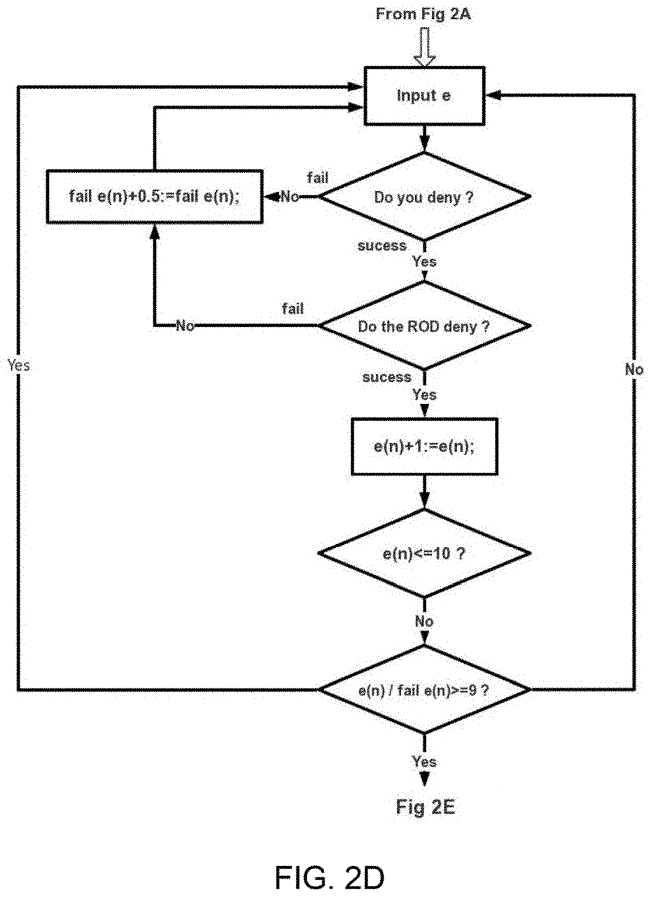

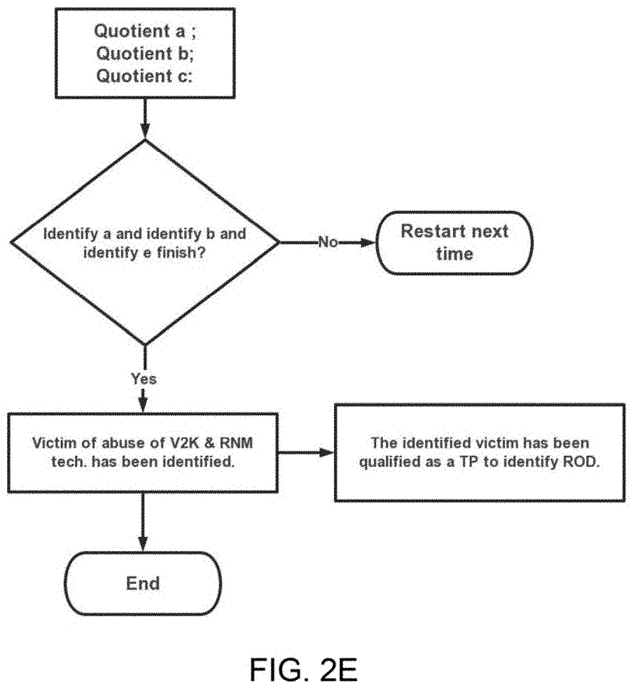

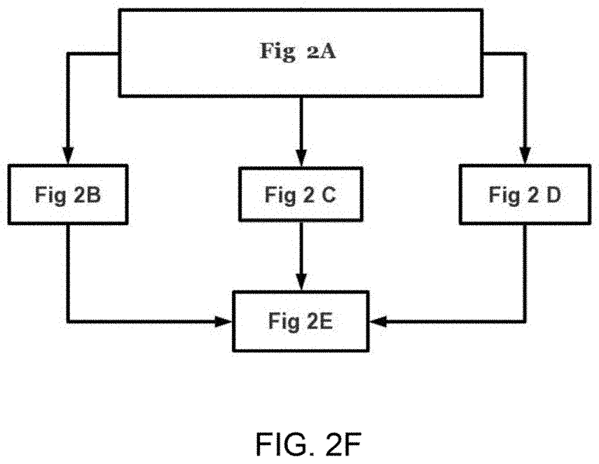

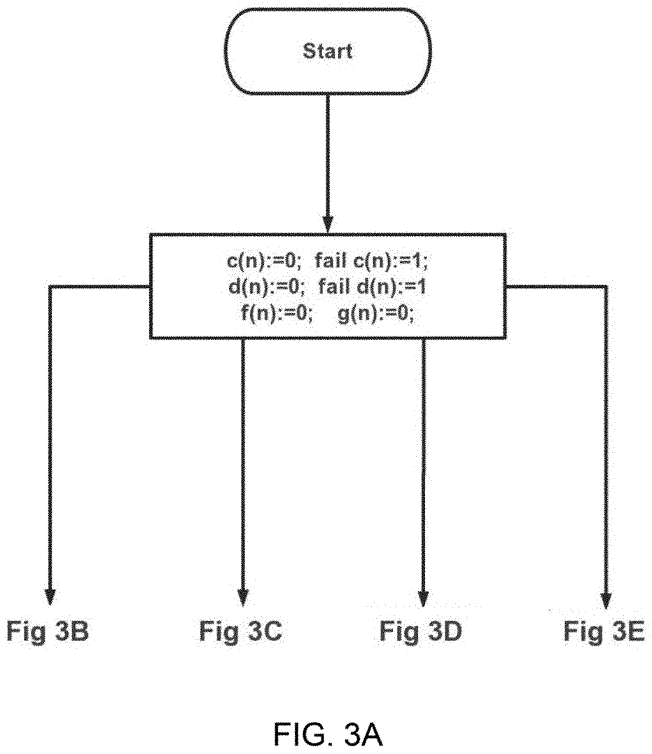

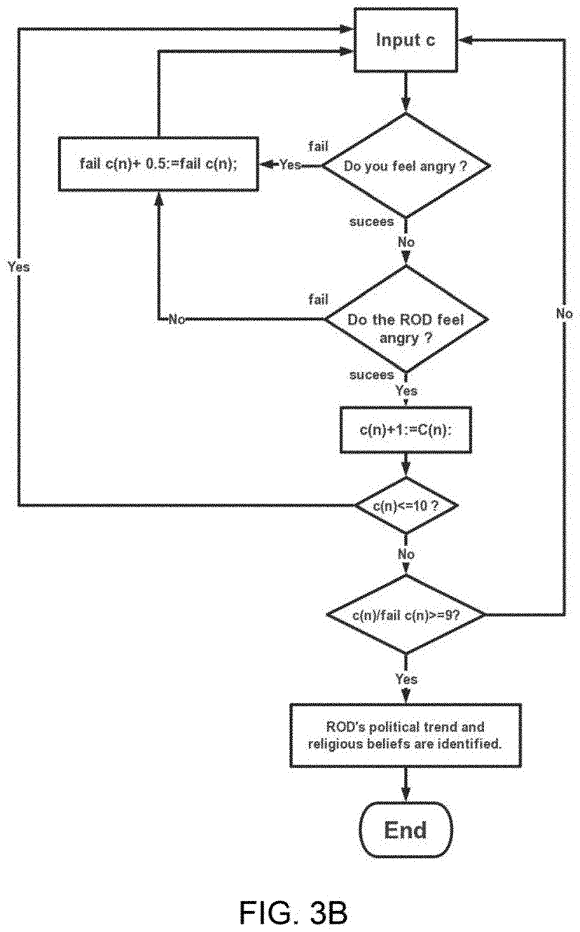

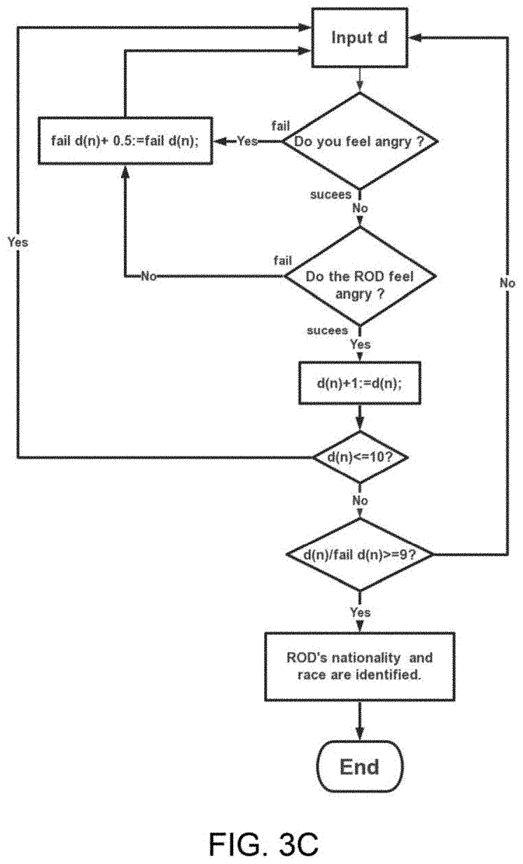

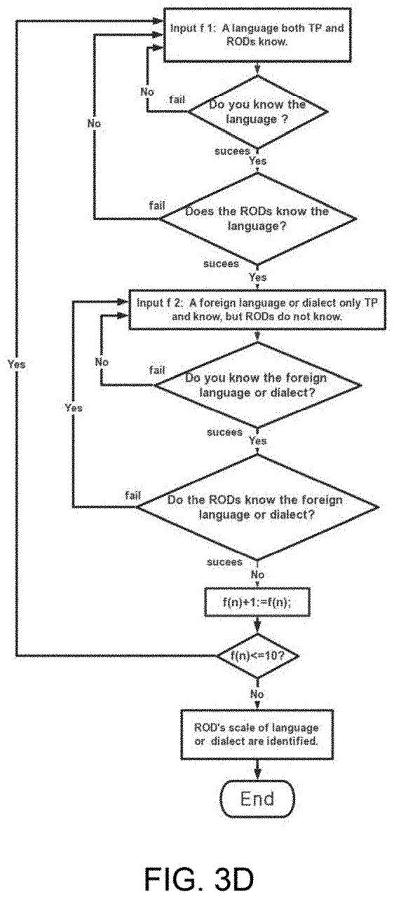

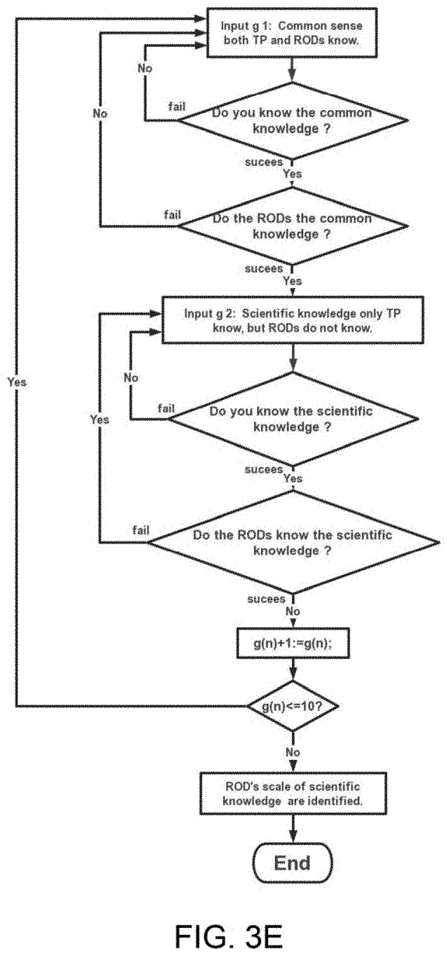

Then the invention use of relatively comparing methods, designed procedures in flowcharts classify emotional responses between the victim's (TP's) and remote operators' (ROD's), designed procedures in flowchart can find out there are significant differences between victim's (TP's) and remote operators' (ROD's) with judgement diamond frames. Use of these difference, computer and controller can identify both victim (TP) and classified identify all kinds status of remote operators (ROD) using devices of V2K and RNM by checking flowcharts FIG. 2 A to FIG. 2F and FIG. 3A to FIG. 3E automatically.

These classified identities including remote operator's (ROD's) nationality, race, political trend, religious belief, organization, scale of language and dialect, scale of scientific knowledge.

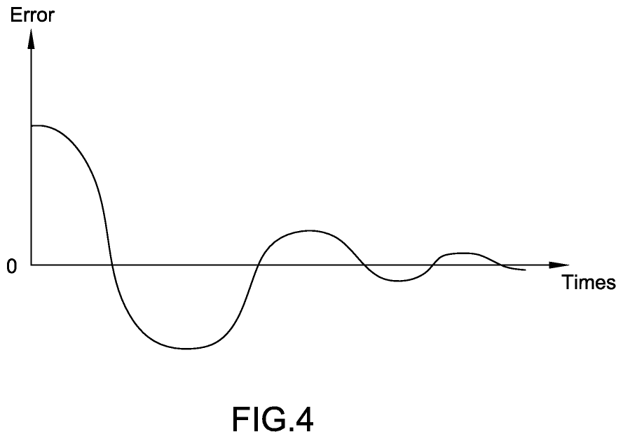

When identifying begin, a identifying curve indicates identifying error changes gradually from large to small and approach to accurate identification at last, the principle is similar with automatic control decay curve, which is shown in FIG. 4.

Such similar curve is widely used for PID controller in automatic control technology which is used to control like temperature and pressure, but the invention uses it to identify victim (TP) and to identify ROD, mainly for identifying ROD.

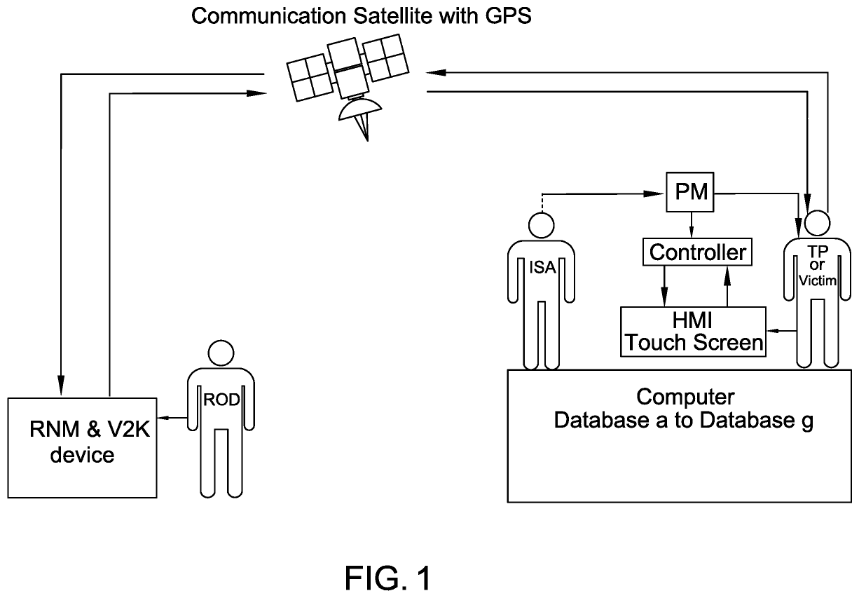

Referring to FIGS. 1-14, the present invention consists six parts:

I. Polygraph Meter: (PM)

There are many kinds of ordinary polygraph meters.

Polygraph meter is used to confirm the tested person's (TP's) answer is true.

If high class digital polygraph which can connects with a computer and sent truth or fails signals automatically is available, the automatic system of identification could run very fast.

Expensive and high accurate instrument is using fMRI (Functional Magnetic Resonance Imagine) when in real need. Illuminating lies with brain scan outshines polygraph test, fMRI spots more lies in first controlled comparison of the two technologies.

II. ISA: an identification system administrator

An identification administrator (ISA) asks a tested person (TP) to choose some special contents from a database in a computer which could cause those remote operator's or attacker's (ROD)'s psychological reaction and response via V2K & RNM device, but cannot cause tested person's (TP) same psychological reaction. Meanwhile, the tested person (TP) to answer YES or NO with pressing designed buttons on HMI according those designed questions contents which also shows on HMI in diamond frames in flowcharts in a computer about attacker's or call remote dives operators' (ROD's) psychological reaction and response which the tested person (TP) can feel or sense, ISA then checks the polygraph meter to confirm TP′ answer is true.

III. Tested person (TP)

Tested person (TP) should claim himself/herself is a victim of abusing V2K & RNM technology. Tested person who is connected to polygraph meter and answer questions which can be analyzed and identify who is the remote attacker or operator (ROD) using V2K & RNM device, furthermore the invention may analyze and identify attacker's or operator's (ROD's) nationality, race, political belief and religious belief, scale of language and dialect, scale of scientific knowledge and which organization by the designed procedures in a computer.

Of course, the qualified tested person (TP) should be emotional stable and rational thinking, as well as cooperative with identification system administrator (ISA).

IV. A computer contains seven classified databases a; b; c; d; e; f and g which classify psychological attacking contents. Every classified database can let tested person (TP) choose contents to launch some classified psychological attacks to ROD to cause ROD's classified psychological responses.

This the ROD's classified psychological responses can be used to identify who is victim (TP) and who is attacker or operator (ROD) by operating programs in flowcharts in a computer and in a controller.

Of course, the classified contents can be updated and increased according different requirements in a computer used in identifying process.

V. A controller: A controller manages logical calculations when automatic system is running. It also contains twelve counters in flowcharts, flowcharts is a programmed software, among them, seven counters are successful counters which record every time successful identification and accumulate the times of successful identification: a(n), b(n), c(n), d(n), e(n), f(n) and g(n). Another five counters are failure counters which record every time failure identification and accumulate the times of identification: fail a(n), fail b(n), fail c(n), fail d(n) and fail e(n).

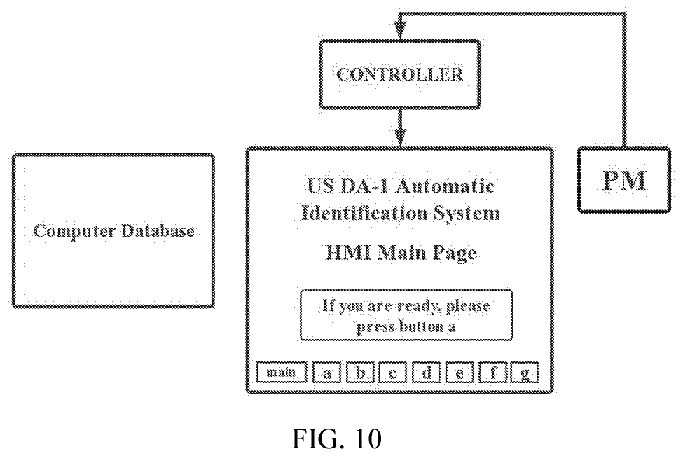

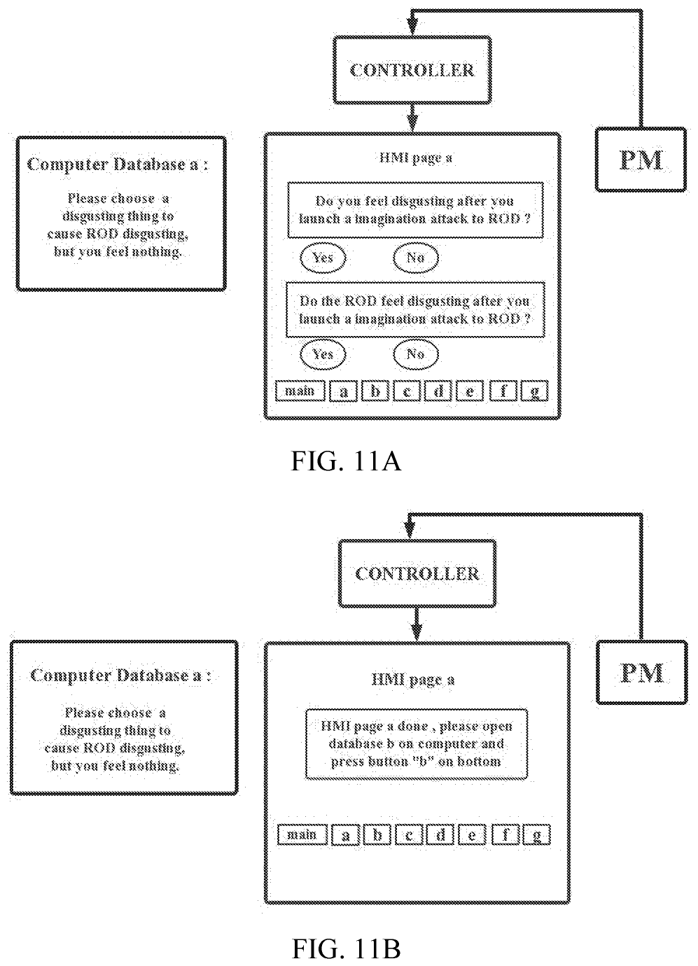

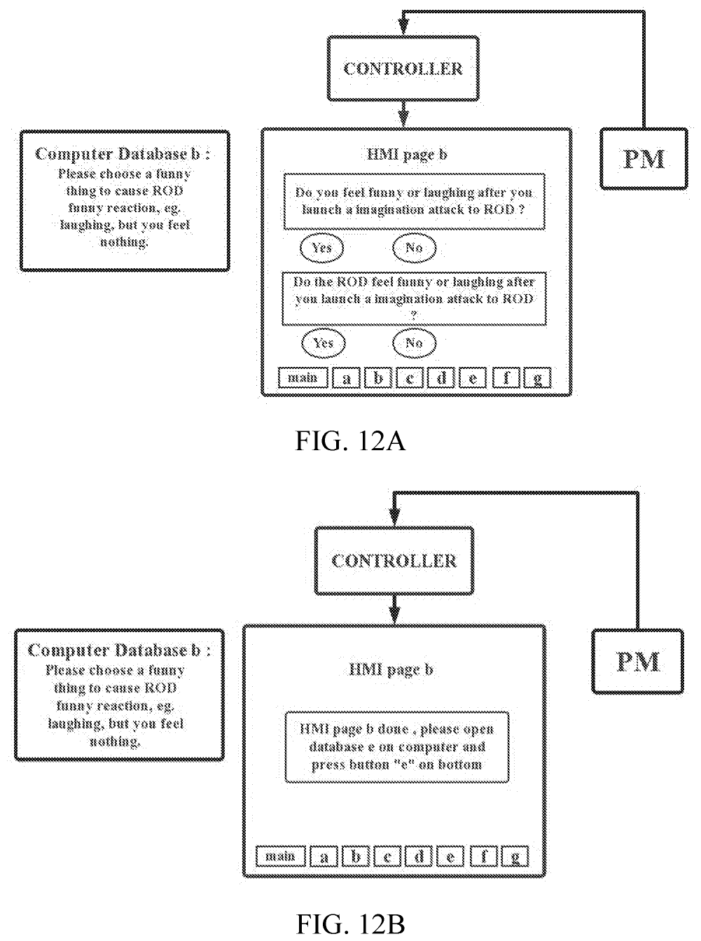

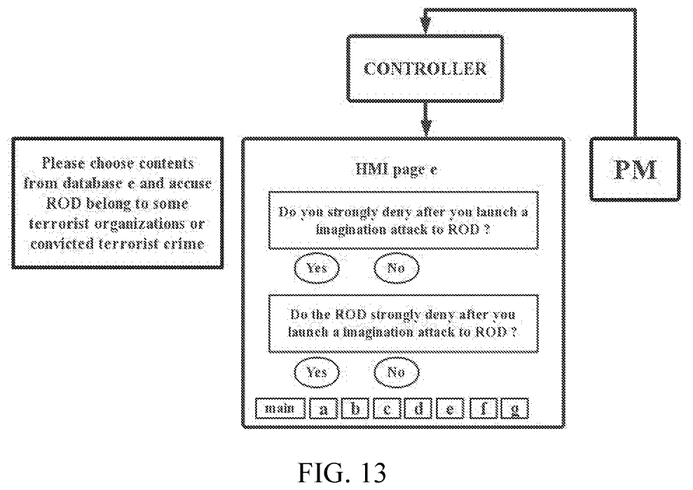

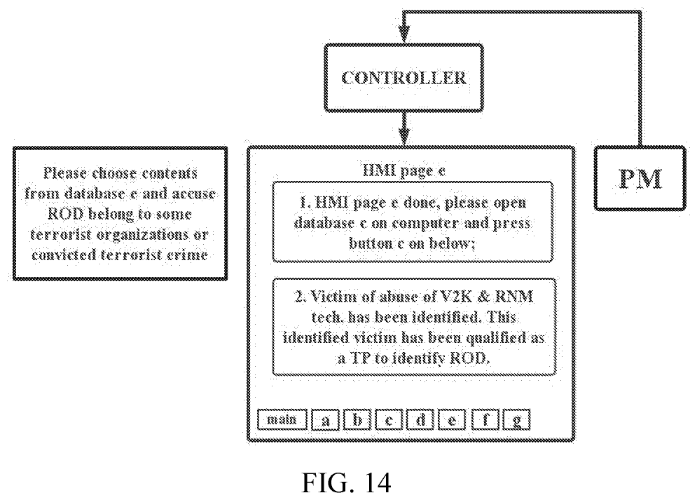

VI. A human-machine interface (HMI), or called touch screen: the touch screen will have eight pages, except the main page FIG. 10, they are FIG. 11A (HMI page a); FIG. 11B (HMI page a done); FIG. 12A (HMI page b); FIG. 12B HMI page b done; FIG. 13 (HMI page e); FIG. 14 (HMI page e done); these Figs have been drawn out, other FIG. 3A to FIG. 3E are similar, so does not been drawn out. Touch screen operating pages represents one section of flowchart shows designed questions to TP, TP also answers questions with pressing “YES” or “NO” buttons on touch screen; Finally, the touch screen also shows result of identifications with English (or other languages) displaying window to TP and IEA. At same time the touch screen connected with computer keep communication and controller with some baud rate. All programs are running in background of HMI touch screen in a controller and a computer. There one main program, eight subroutines and one interrupt. Main program calls subroutine when in need. An interrupt program is triggered when PM check for a lie.

Judgement contains in every diamond in flowcharts, TP answers questions in every diamond frame which shows on HMI touch screen and a polygraph meter to confirm if TP's every answer is true.

Every diamond frame have two flow lines directions, word “success” beside the line indicates one time of successful identification; word “fail” beside the line indicates one time of failure identification.

There are two sets of flowcharts which are programmed software in a controller.

The first set of flowcharts consist of FIG. 2A to FIG. 2F which are used to identify a victim (TP) of abuse technology of V2K & RNM, the input contents from database a, b and e; there are three successful counters: a(n), b(n) and e(n), there are also three fail counters: fail a(n), fail b(n) and fail e(n).

Among these fail counters, they represent any failure identification, fail a(n), fail b(n), fail e(n) in first set of flowcharts; but fail counters fail c(n) and fail d(n) in second set of flowcharts.

In the identifying process, the number in successful counters is divided by the number in fail counters, if the quotient larger or equal to 9, the identification has been completed. In case of need, the quotient can be larger than 9.

In order to avoiding the divisor (denominator of the score) is zero, we assign an initial value 1 to every fail counter. In the fractional number in a(n), b(n), c(n), d(n) and e(n) are numerator, but the number in fail a(n), fail b(n), fail c(n), fail d(n) and fail e(n) are denominator.

The second set of flowcharts consist of FIG. 3A to FIG. 3E, which are used to identify those remote operators' or attackers' (ROD's) all kinds status, who are using devices of V2K & RNM, the input contents from database c, d, f and g.

There are also two successful counters: c(n) and d(n), two fail counters: fail c(n) and fail d(n), but there are not fail counter fail f (n) and fail g(n).

Each flowchart input contents according type and characteristics from contents database in a computer, ISA asks TP to choose some suitable contents form the database to launch a psychological thoughts attack to ROD, then TP answer questions which show on HMI in diamond frame in flowcharts about what kind of psychological response from ROD which TP can feel and sense. Seven category of input contents are in seven classified databases.

Every database stores different content like following, but these contents in different databases inputs classified content only the method put into using. For examples, database of category d can input twenty five names of countries and more different races, because it was said there were twenty five countries own the technology of remote neutral monitoring & voice to skull or relevant equipment. So ISA can store these names of twenty five countries and their races in database d of a computer when the method in operating.

After TP answers the questions, ISA checks PM to confirm if the answer is true.

Seven category of input contents in seven classified databases, each database stores different content like following, but these contents in different databases store classified contents only the method put into running.

Category a:

The category a database contains a lot of contents which are specifically causing remote operator's (ROD's) using devices of V2K & RNM disgusting psychological response, but they will not cause tested person's (TP) disgusting psychological reaction, because the identification system administrator (ISA) lets tested person (TP) himself/herself choose some disgusting contents in this database intentionally to cause ROD disgusting psychological response only, but does not cause the tested person (TP) himself/herself disgusting psychological reaction.

After TP has chosen a kind of disgusting content from database a, he/she must follow instructions in the computer to launch a thoughts imagination psychological attack to ROD and then answer question in diamond frame in flowchart which shows on HMI touch screen, ISA then checks PM to confirm TP's answer is true.

According automatic control theory, multiple TP's disgusting psychological attacking cycles will generate precise identification: the disgusting psychological response comes from ROD, but does not belong to TP.

For example, in the database, TP find a picture which describe “the fresh stinky stool is pulled out by me and sent to ROD as breakfast with a picture of plate of stool.

It is common sense nearly everyone never feels disgusting facing himself/herself fresh stinky stool which just pulled out, but everyone may feel very disgusting to see others fresh stinky stool in short distance. This psychological experiment has been confirmed in some Chinese victims who imagine showing himself/herself fresh stinky stool for those remote operators (RODs) using devices of V2K & RNM, then caused disgusting feeling from those remote operators (RODs) using devices of V2K & RNM which these victims can feel and sense. This experiment can be repeatedly confirmed to same result.

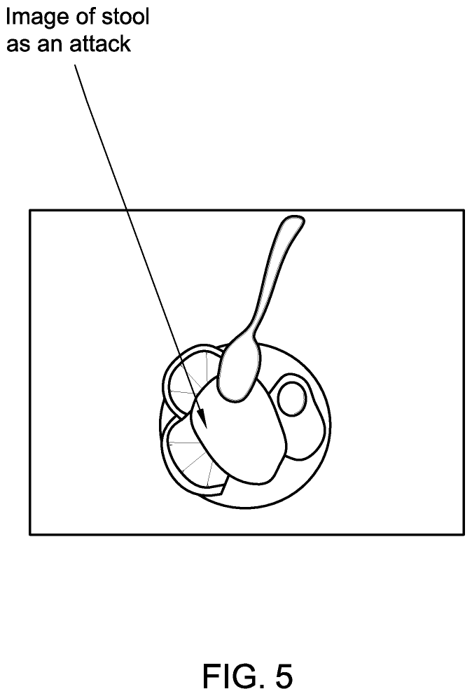

FIG. 2A to FIG. 2F illustrates a first phase of flowchart. FIG. 5 illustrates examples of disgusting things.

Category b:

Category b is used for identifying a TP who is a victim of abuse technology of RNM & V2K.

The category b database contents which are specifically causing remote operator (ROD) using devices of V2K & RNM funny psychological response. E.g., LAUFHING, but they will not cause tested person's (TP's) funny psychological reaction, because the identification system administrator (ISA) lets tested person (TP) himself/herself choose some funny stories or humor contents in this database intentionally only to cause ROD's funny psychological response, but does not cause the tested person (TP) himself/herself funny psychological reaction. For example, the host of a talk show makes audience laugh, but the joke player himself/herself does not laugh.

According UK English learning book “New Conception English: third set book text 29 “Funny or Not?” which described people in indifferent country and different race have different humor sense.

Example of a funny story:

“When my five years old son was ‘newly’ potty trained, I was using the restroom at a supermarket. He gets the toilet paper for me and when I say ‘thank you’ and try to take it from him. He says (loudly), “No, Mommy, bend over and I will wipe your butt.” He was so proud of himself for trying to help me. My face was red and I could hear chuckles all around me.”

After TP has chosen a kind of funny content from database a, he/she must flow instruction in a computer to launch a thoughts imagination psychological attack to RODs and then answer question in diamond frame in flowchart, which shows on touch screen, ISA checks PM to confirm TP's answer is true.

According automatic control theory, multiple TP's funny psychological attacking cycles will generate precise identification: the funny psychological response comes from ROD but does not belong to TP's.

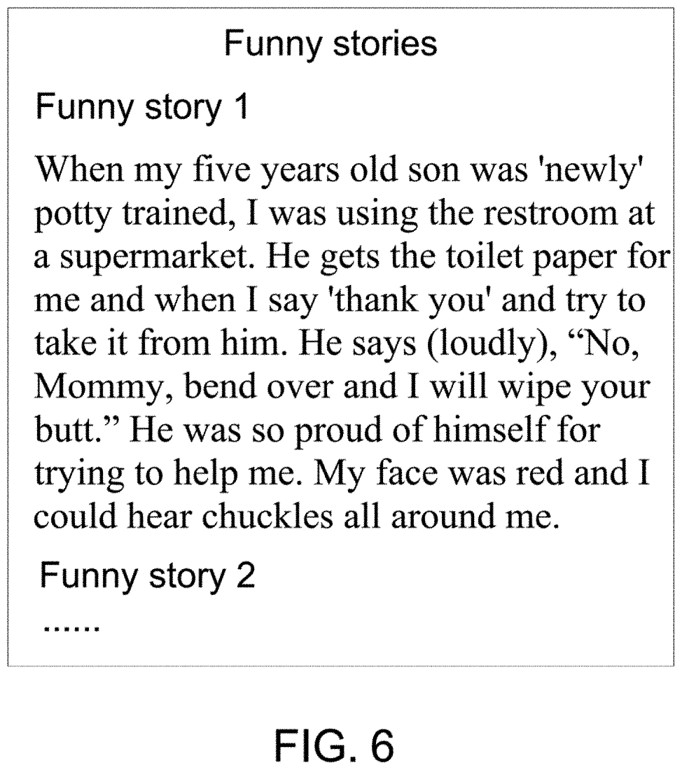

FIG. 2A to FIG. 2F illustrates a first phase of flowchart. FIG. 6 illustrates example of funny stories.

Category c:

Category c is used for identifying ROD's political trend and religious belief.

The category c database contains all kinds of anti-political trend and anti-religious belief are specifically causing remote operator's (ROD's) using devices of V2K & RNM angry psychological response: angry curse and refutation, but they will not cause tested person's (TP's) angry psychological reaction, because the identification system administrator (ISA) lets tested person (TP) himself/herself choose some contents form database which fiercely oppose ROD″s political trends or contents which seriously insult ROD's religious beliefs in this database intentionally only to cause ROD's angry psychological response, but does not cause the tested person (TP) himself/herself angry psychological reaction.

### Example 1

TP chooses one of these political party's symbols which RODs belong to the political party from database c to attack it with brain thinking imagination with stinky stool stain political symbols or burn political symbols etc. to cause ROD's antipathy and angry which TP can feel clearly.

### Example 2

TP chooses one of these religious symbols which RODs belong to the religious organization from database c to attack it with brain thinking imagination with stinky stool to stain the religious symbols and burn religious book etc. to cause ROD's antipathy and angry which TP can feel clearly.

There is a more very clear example: we assume the TP is not a Communist Party member, but ROD is a Communist Party member who thinks that the Communist Party flag and Communist Party emblem are sacred and inviolable. Then we ask TP to insult the Communist Party flag and Communist Party emblem with stinky stool to stain it or burn them, in this case, TP in the invention system will feel and sense ROD's angry, we are sure ROD is a Communist Party member, because people who are not Communist Party member feel nothing insulting the Communist Party flag and Communist Party emblem in such way, only a Communist Party member can feel angry, so there is not any psychologist can deny the accuracy of the invention of identification.

After TP has chosen a kind of content from database c, he/she must follow instruction in a computer to launch a thoughts imagination psychological attack to ROD and then answer question in diamond frame in flowchart which shows on touch screen, then ISA checks PM to confirm TP's answer is true.

According automatic control theory, multiple TP's political or religious psychological attacking cycles will generate precise identification: the angry psychological response comes from RODs because of political or religious reasons, but does not belong to TP's.

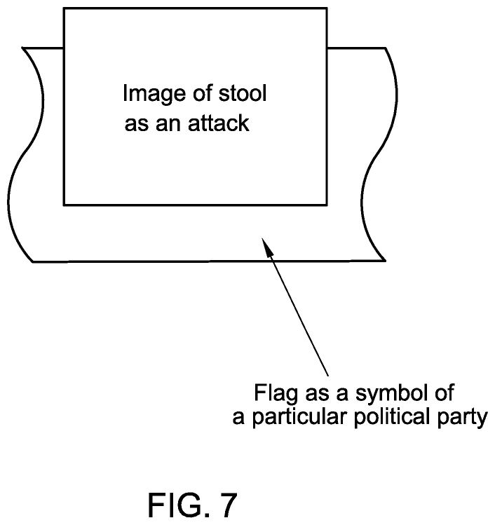

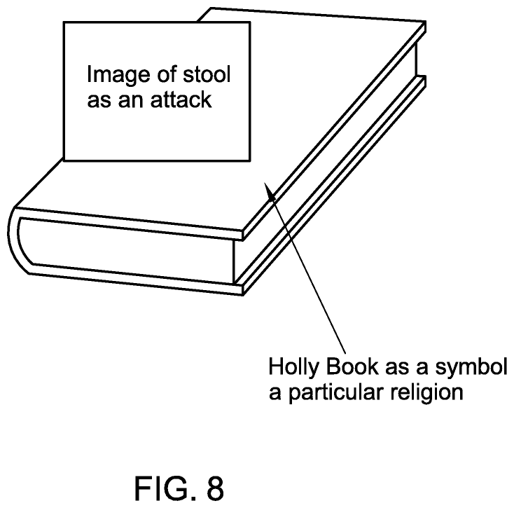

FIG. 3A to FIG. 3E illustrates a second phase of flowchart. FIG. 7 illustrates examples of a man pulling a large pile of stool on a flag of a political party. FIG. 8 illustrates examples of a man pulling a large pile of stool on a sacred sign of religion. FIG. 8

Category d:

Category d is used for identifying ROD's nationality and race.

The category d database contents are different countries and different races which specifically cause remote operator's (ROD's) using devices of V2K & RNM angry psychological response: angry curse, because the identification system administrator (ISA) lets tested person (TP) himself/herself choose some contents which destroy ROD's country and ROD's race in this database intentionally only to cause ROD angry psychological response, but does not cause the tested person (TP) himself/herself angry psychological reaction.

For example, TP choose some nuclear explosion videos in database d and meanwhile use thoughts to tell ROD: “I hope to use nuclear weapon to destroy your country and kill all your family members, let your ROD's body and corpse became vapor or steam in high temperature, kill all your race from the earth”, such psychological attack is sure to cause ROD's angry response which TP can feel and sense. Once ROD's angry is identified, ROD's nationality and race have been identified.

For example: TP can assume imagining huge earthquake destroying a country video choosing from computer database to do a country where RODs live to cause ROD's antipathy and angry which TP can feel clearly.

For example: TP can assume imagining huge tsunami drowning a country video choosing from computer database to launch a psychological attack a country where RODs live to cause ROD's antipathy and angry which TP can feel clearly.

After TP has chosen a kind of content from database c, he/she must follow instruction in a computer to launch a thoughts imagination psychological attack to ROD and then answer question in diamond frame in flowchart which shows on touch screen then ISA checks PM to confirm TP's answer is true.

According automatic control theory, multiple TP's nationality or race psychological attacking cycles will generate precise identification: the angry psychological response comes from ROD, because ROD's nationality or race are attacked and ROD are infuriated, but these does not belong to TP's.

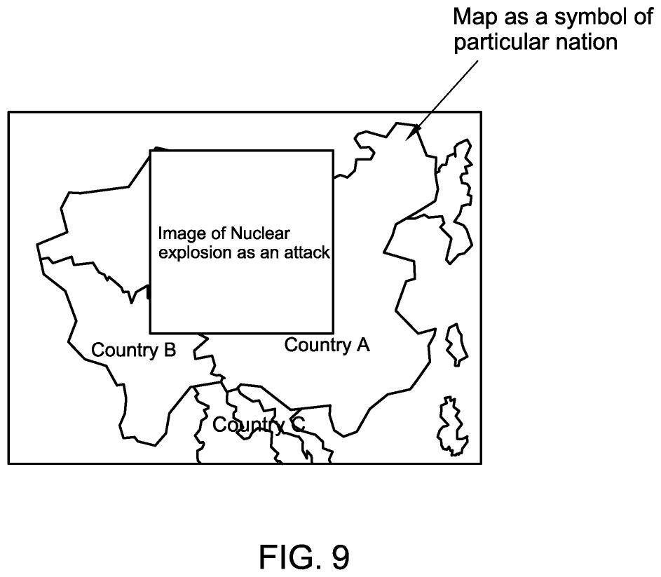

FIG. 3A to FIG. 3 E illustrates a second phase of flowchart. FIG. 9 illustrates examples of a mushroom cloud of nuclear explosion rises on the land of a particular country.

Category e:

Category e is used for identifying a TP is a victim of abuse technology of RNM & V2K.

The category e database contents with all kinds of foreign terrorist organizations are specifically causing remote operator's using devices of V2K & RNM ROD's angry psychological response: strongly deny, because the identification system administrator (ISA) lets tested person (TP) himself/herself choose some contents which accuse RODs committed a terrorist crime, such as terrorist attack on United States and RODs belong to some terrorist organization to cause ROD's strongly deny which TP can feel and sense. Because everyone knows any terrorist who attacked USA, US government must eliminate the terrorist in anywhere in the world.

On the other hand, TP is not a terrorist and never commit felony, so TP does not need to deny or admit.

So once RODs make a strongly deny, it is sure the response from RODs and it is sure the response is not auditory hallucination belong the victim (TP). This is an important step to identify a victim of abusing technology of V2K & RNM.

After TP has chosen a kind of content from database e, he/she must follow instruction in a computer to launch a thoughts imagination psychological attack to ROD and then answer question in diamond frame in flowchart which shows on touch screen, then ISA checks PM to confirm TP's answer is true.

According automatic control theory, multiple TP's accusing ROD's belong terrorist organizations or convicted terrorist crime psychological attacking cycles will generate precise identification: the strongly denying psychological response comes from ROD, but these does not belong to TP's.

FIG. 2A to FIG. 2F illustrates a first phase of flowchart.

Foreign organizations will be searched from time to time and added into the database e by ISA when in need.

Category f:

Category f is used for identifying ROD's scale of language and dialect.

The category f database contents with different languages and different dialects are specifically causing remote operator's (ROD's) using devices of V2K & RNM slow down psychological response: lighten harassment or stop harassment, because the identification system administrator (ISA) lets tested person (TP) intentionally imagine something using a foreign language or dialect which ROD do not know. The reason why ROD lighten harassment or stop harassment when they meet some foreign language or dialect which they do not know, these ROD need time to understand and consider how response on their device of V2K & RNM, so these ROD loosen or stop their harassment to TP's brain temporarily.

For example, we assume those ROD don't understand English, only know Chinese language, in such case, TP uses English to consider something, or even TP reads an English article, at this point, TP can find the harassment form ROD lighten or stop, the reason is those ROD don't understand what TP is considering or reading, so those ROD's reaction on their devices gets slow or stop temporarily.

Another example, we assume TP can speak both dialects Cantonese and Mandarin, but those ROD only can speak and understand Mandarin, in this case, TP use Cantonese consider something, TP also can find the harassment form ROD lighten or stop, the reason is those ROD don't understand what TP is considering with Cantonese, so those ROD's reaction on their devices get slow or stop temporarily.

After TP has chosen a kind of content from database f, he/she must follow instruction in a computer to launch a thoughts imagination psychological attack to ROD and then answer question in diamond frame in flowchart which shows on touch screen, then ISA checks PM to confirm TP's answer is true.

According automatic control theory, multiple TP's psychological attacking cycles with languages or dialect will generate precise identification: the harassment become lighten or stops response comes from ROD, but these does not belong to TP's.

FIG. 3A to FIG. 3E illustrate a second phase of flowchart.

Table 1 shows examples of languages spoken in different countries of the world.

TABLE 1
Languages spoken in different countries of the world

| | Languages spoken (the percent of the population that |
|---|---|
| Country | speaks a particular language is also given, if available) |
| Afghanistan | Dari Persian, Pashtu (both official), other Turkic |
| | and minor languages |
| Albania | Albanian (Tosk is the official dialect), Greek |
| Algeria | Arabic (official), French, Berber dialects |
| Andorra | Catalán (official), French, Castilian, Portuguese |
| Angola | Portuguese (official), Bantu and other African languages |
| Antigua and | English (official), local dialects |
| Barbuda | |
| Argentina | Spanish (official), English, Italian, German, French |
| Armenia | Armenian 98%, Yezidi, Russian |
| Australia | English 79%, native and other languages |
| Austria | German (official nationwide); Slovene, Croatian, Hungarian |
| | (each official in one region) |
| Azerbaijan | Azerbaijani Turkic 89%, Russian 3%, Armenian 2%, other |
| | 6% (1995 est.) |
| Bahamas | English (official), Creole (among Haitian immigrants) |
| Bahrain | Arabic, English, Farsi, Urdu |
| Bangladesh | Bangla (official), English |
| Barbados | English |
| Belarus | Belorussian (White Russian), Russian, other |
| Belgium | Dutch (Flemish) 60%, French 40%, German less than 1% |
| | (all official) |
| Belize | English (official), Spanish, Mayan, Garifuna (Carib), |
| | Creole |
| Benin | French (official), Fon, Yoruba, tribal languages |
| Bhutan | Dzongkha (official), Tibetan dialects (among Bhotes), |
| | Nepalese dialects (among Nepalese) |
| Bolivia | Spanish, Quechua, Aymara (all official) |
| Bosnia and | Bosnian, Croatian, Serbian |
| Herzegovina | |
| Botswana | English 2% (official), Setswana 78%, Kalanga 8%, |
| | Sekgalagadi 3%, other (2001) |
| Brazil | Portuguese (official), Spanish, English, French |
| Brunei | Malay (official), English, Chinese |
| Bulgaria | Bulgarian 85%, Turkish 10%, Roma 4% |
| Burkina Faso | French (official); native African (Sudanic) languages 90% |
| Burundi | Kirundi and French (official), Swahili |
| Cambodia | Khmer 95% (official), French, English |
| Cameroon | French, English (both official); 24 major African language |
| | groups |
| Canada | English 59.3%, French 23.2% (both official); other 17.5% |
| Cape Verde | Portuguese, Criuolo |
| Central African | French (official), Sangho (lingua franca, national), |
| Republic | tribal languages |
| Chad | French, Arabic (both official); Sara; more than 120 |
| | languages and dialects |
| Chile | Spanish |
| China | Standard Chinese (Mandarin/Putonghua), Yue (Cantonese), |
| | Wu (Shanghaiese), Minbei (Fuzhou), Minnan (Hokkien- |
| | Taiwanese), Xiang, Gan, Hakka dialects, minority languages |
| Colombia | Spanish |
| Comoros | Arabic and French (both official), Shikomoro |
| | (Swahili/Arabic blend) |
| Congo, Democratic | French (official), Lingala, Kingwana, Kikongo, Tshiluba |
| Republic of the | |
| Congo, Republic of | French (official), Lingala, Monokutuba, Kikongo, many |
| | local languages and dialects |
| Costa Rica | Spanish (official), English |
| Côte d′Ivoire | French (official) and African languages (Dioula esp.) |
| Croatia | Croatian 96% (official), other 4% (including Italian, |
| | Hungarian, Czech, Slovak, German) |
| Cuba | Spanish |
| Cyprus | Greek, Turkish (both official); English |
| Czech Republic | Czech |
| Denmark | Danish, Faroese, Greenlandic (Inuit dialect), German; |
| | English is the predominant second language |
| Djibouti | French and Arabic (both official), Somali, Afar |
| Dominica | English (official) and French patois |
| Dominican Republic | Spanish |
| East Timor | Tetum, Portuguese (official); Bahasa Indonesia, English; |
| | other indigenous languages, including Tetum, Galole, |
| | Mambae, and Kemak |
| Ecuador | Spanish (official), Quechua, other Amerindian languages |
| Egypt | Arabic (official), English and French widely |
| | understood by educated classes |
| El Salvador | Spanish, Nahua (among some Amerindians) |
| Equatorial Guinea | Spanish, French (both official); pidgin English, Fang, Bubi, |
| | Ibo |
| Eritrea | Afar, Arabic, Tigre and Kunama, Tigrinya, other Cushitic |
| | languages |
| Estonia | Estonian 67% (official), Russian 30%, other (2000) |
| Ethiopia | Amharic, Tigrigna, Orominga, Guaragigna, Somali, Arabic, |
| | English, over 70 others |
| Fiji | English (official), Fijian, Hindustani |
| Finland | Finnish 92%, Swedish 6% (both official); small Sami- |
| | (Lapp) and Russian-speaking minorities |
| France | French 100%, rapidly declining regional dialects (Provençal, |
| | Breton, Alsatian, Corsican, Catalan, Basque, Flemish) |
| Gabon | French (official), Fang, Myene, Nzebi, Bapounou/Eschira, |
| | Bandjabi |
| Gambia | English (official), Mandinka, Wolof, Fula, other indigenous |
| Georgia | Georgian 71% (official), Russian 9%, Armenian 7%, |
| | Azerbaijani 6%, other 7% (Abkhaz is the official language in |
| | Abkhazia) |
| Germany | German |
| Ghana | English (official), African languages (including Akan, |
| | Moshi-Dagomba, Ewe, and Ga) |
| Greece | Greek 99% (official), English, French |
| Grenada | English (official), French patois |
| Guatemala | Spanish 60%, Amerindian languages 40% (23 officially |
| | recognized Amerindian languages, including Quiche, |
| | Cakchiquel, Kekchi, Mam, Garifuna, and Xinca) |
| Guinea | French (official), native tongues (Malinké, Susu, Fulani) |
| Guinea-Bissau | Portuguese (official), Criolo, African languages |
| Guyana | English (official), Amerindian dialects, Creole, Hindi, Urdu |
| Haiti | Creole and French (both official) |
| Honduras | Spanish (official), Amerindian dialects; English widely |
| | spoken in business |
| Hungary | Magyar (Hungarian) 94%, other 6% |
| Iceland | Icelandic, English, Nordic languages, German widely |
| | spoken |
| India | Hindi 30%, English, Bengali, Gujarati, Kashmiri, |
| | Malayalam, Marathi, Oriya, Punjabi, Tamil, Telugu, Urdu, |
| | Kannada, Assamese, Sanskrit, Sindhi (all official); |
| | Hindi/Urdu; 1,600+ dialects |
| Indonesia | Bahasa Indonesia (official), English, Dutch, Javanese, and |
| | more than 580 other languages and dialects |
| Iran | Persian and Persian dialects 58%, Turkic and Turkic dialects |
| | 26%, Kurdish 9%, Luri 2%, Balochi 1%, Arabic 1%, |
| | Turkish 1%, other 2% |
| Iraq | Arabic (official), Kurdish (official in Kurdish regions), |
| | Assyrian, Armenian |
| Ireland | English, Irish (Gaelic) (both official) |
| Israel | Hebrew (official), Arabic, English |
| Italy | Italian (official); German-, French-, and Slovene-speaking |
| | minorities |
| Jamaica | English, Jamaican Creole |
| Japan | Japanese |
| Jordan | Arabic (official), English |
| Kazakhstan | Kazak (Qazaq, state language) 64%; Russian (official, used |
| | in everyday business) 95% (2001 est.) |
| Kenya | English (official), Swahili (national), and numerous |
| | indigenous languages |
| Kiribati | English (official), I-Kiribati (Gilbertese) |
| Korea, North | Korean |
| Korea, South | Korean, English widely taught |
| Kosovo | Albanian (official), Serbian (official), Bosnian, Turkish, |
| | Roma |
| Kuwait | Arabic (official), English |
| Kyrgyzstan | Kyrgyz, Russian (both official) |
| Laos | Lao (official), French, English, various ethnic languages |
| Latvia | Latvian 58% (official), Russian 38%, Lithuanian, other |
| | (2000) |
| Lebanon | Arabic (official), French, English, Armenian |
| Lesotho | English, Sesotho (both official); Zulu, Xhosa |
| Liberia | English 20% (official), some 20 ethnic-group languages |
| Libya | Arabic, Italian, and English widely understood in major |
| | cities |
| Liechtenstein | German (official), Alemannic dialect |
| Lithuania | Lithuanian 82% (official), Russian 8%, Polish 6% (2001) |
| Luxembourg | Luxermbourgish (national) French, German (both |
| | administrative) |
| Macedonia | Macedonian 67%, Albanian 25% (both official); Turkish |
| | 4%, Roma 2%, Serbian 1% (2002) |
| Madagascar | Malagasy and French (both official) |
| Malawi | Chichewa 57.2% (official), Chinyanja 12.8%, Chiyao |
| | 10.1%, Chitumbuka 9.5%, Chisena 2.7%, Chilomwe 2.4%, |
| | Chitonga 1.7%, other 3.6% (1998) |
| Malaysia | Bahasa Melayu (Malay, official), English, Chinese dialects |
| | (Cantonese, Mandarin, Hokkien, Hakka, Hainan, Foochow), |
| | Tamil, Telugu, Malayalam, Panjabi, Thai; several |
| | indigenous languages (including Iban, Kadazan) in East |
| | Malaysia |
| Maldives | Maldivian Dhivehi (official); English spoken by most |
| | government officials |
| Mali | French (official), Bambara 80%, numerous African |
| | languages |
| Malta | Maltese and English (both official) |
| Marshall Islands | Marshallese 98% (two major dialects from the Malayo- |
| | Polynesian family), English widely spoken as a second |
| | language (both official); Japanese |
| Mauritania | Hassaniya Arabic (official), Pulaar, Soninke, French, Wolof |
| Mauritius | English less than 1% (official), Creole 81%, Bojpoori 12%, |
| | French 3% (2000) |
| Mexico | Spanish, various Mayan, Nahuatl, and other regional |
| | indigenous languages |
| Micronesia | English (official, common), Chukese, Pohnpeian, Yapase, |
| | Kosrean, Ellithian, Woleaian, Nukuoro, Kapingamarangi |
| Moldova | Moldovan (official; virtually the same as Romanian), |
| | Russian, Gagauz (a Turkish dialect) |
| Monaco | French (official), English, Italian, MonEgasque |
| Mongolia | Mongolian, 90%; also Turkic and Russian (1999) |
| Montenegro | Serbian/Montenegrin (Ijekavian dialect-official) |
| Morocco | Arabic (official), Berber dialects, French often used for |
| | business, government, and diplomacy |
| Mozambique | Portuguese 9% (official; second language of 27%), |
| | Emakhuwa 26%, Xichangana 11%, Elomwe 8%, Cisena 7%, |
| | Echuwabo 6%, other Mozambican languages 32% (1997) |
| Myanmar | Burmese, minority languages |
| Namibia | English 7% (official), Afrikaans is common language of |
| | most of the population and of about 60% of the white |
| | population, German 32%; indigenous languages: |
| | Oshivambo, Herero, Nama |
| Nauru | Nauruan (official), English |
| Nepal | Nepali 48% (official), Maithali 12%, Bhojpuri 7%, Tharu |
| | 6%, Tamang 5%, others. English spoken by many in |
| | government and business (2001) |
| Netherlands | Dutch, Frisian (both official) |
| New Zealand | English, Maori (both official) |
| Nicaragua | Spanish 98% (official); English and indigenous languages on |
| | Atlantic coast (1995) |
| Niger | French (official), Hausa, Djerma |
| Nigeria | English (official), Hausa, Yoruba, Ibo, Fulani, and more than |
| | 200 others |
| Norway | Bokmål Norwegian, Nynorsk Norwegian (both official); |
| | small Sami- and Finnish-speaking minorities (Sami is |
| | official in six municipalities) |
| Oman | Arabic (official), English, Baluchi, Urdu, Indian dialects |
| Pakistan | Urdu 8%, English (both official); Punjabi 48%, Sindhi 12%, |
| | Siraiki (a Punjabi variant) 10%, Pashtu 8%, Balochi 3%, |
| | Hindko 2%, Brahui 1%, Burushaski, and others 8% |
| Palau | Palauan 64.7%, English 9.4%, Sonsoralese, Tobi, Angaur |
| | (each official on some islands), Filipino 13.5%, Chinese |
| | 5.7%, Carolinian 1.5%, Japanese 1.5%, other Asian 2.3%, |
| | other languages 1.5% (2000) |
| Palestinian State | Arabic, Hebrew, English |
| (proposed) | |
| Panama | Spanish (official), English 14%, many bilingual |
| Papua New | Tok Pisin (Melanesian Pidgin, the lingua franca), Hiri Motu |
| Guinea | (in Papua region), English 1%-2%; 715 indigenous |
| | languages |
| Paraguay | Spanish, Guarani (both official) |
| Peru | Spanish, Quechua (both official); Aymara; many minor |
| | Amazonian languages |
| Philippines | Filipino (based on Tagalog), English (both official); eight major |
| | dialects: Tagalog, Cebuano, Ilocano, Hiligaynon or |
| | Ilonggo, Bicol, Waray, Pampango, and Pangasinense |
| Poland | Polish 98% (2002) |
| Portugal | Portuguese (official), Mirandese (official, but locally used) |
| Qatar | Arabic (official); English a common second language |
| Romania | Romanian (official), Hungarian, German |
| Russia | Russian, others |
| Rwanda | Kinyarwanda, French, and English (all official); Kiswahili in |
| | commercial centers |
| St. Kitts and | English |
| Nevis | |
| St. Lucia | English (official), French patois |
| St. Vincent and | English, French patois |
| the Grenadines | |
| Samoa | Samoan, English |
| San Marino | Italian |
| São Tomé and | Portuguese (official) |
| Príncipe | |
| Saudi Arabia | Arabic |
| Senegal | French (official); Wolof, Pulaar, Jola, Mandinka |
| Serbia | Serbian (official); Romanian, Hungarian, Slovak, and |
| | Croatian (all official in Vojvodina); Albanian (official in |
| | Kosovo) |
| Seychelles | Seselwa Creole 92%, English 5%, French (all official) |
| | (2002) |
| Sierra Leone | English (official), Mende (southern vernacular), Temne |
| | (northern vernacular), Krio (lingua franca) |
| Singapore | Mandarin 35%, English 23%, Malay 14.1%, Hokkien |
| | 11.4%, Cantonese 5.7%, Teochew 4.9%, Tamil 3.2%, other |
| | Chinese dialects 1.8%, other 0.9% (2000) |
| Slovakia | Slovak 84% (official), Hungarian 11%, Roma 2%, Ukrainian |
| | 1% (2001) |
| Slovenia | Slovenian 91%, Serbo-Croatian 5% (2002) |
| Solomon Islands | English 1%-2% (official), Melanesian pidgin (lingua |
| | franca), 120 indigenous languages |
| Somalia | Somali (official), Arabic, English, Italian |
| South Africa | IsiZulu 23.8%, IsiXhosa 17.6%, Afrikaans 13.3%, Sepedi |
| | 9.4%, English 8.2%, Setswana 8.2%, Sesotho 7.9%, |
| | Xitsonga 4.4%, other 7.2% |
| South Sudan | English (official), Arabic (includes Juba and Sudanese |
| | variants) (official), regional languages include Dinka, Nuer, |
| | Bari, Zande, Shilluk |
| Spain | Castilian Spanish 74% (official nationwide); Catalan 17%, |
| | Galician 7%, Basque 2% (each official regionally) |
| Sri Lanka | Sinhala 74% (official and national), Tamil 18% (national), |
| | other 8%; English is commonly used in government and |
| | spoken competently by about 10% |
| Sudan | Arabic (official), Nubian, Ta Bedawie, diverse dialects of |
| | Nilotic, Nilo-Hamitic, Sudanic languages, English |
| Suriname | Dutch (official), Surinamese (lingua franca), English widely |
| | spoken, Hindustani, Javanese |
| Swaziland | English, siSwati (both official) |
| Sweden | Swedish, small Sami- and Finnish-speaking minorities |
| Switzerland | German 64%, French 20%, Italian 7% (all official); |
| | Romansch 0.5% (national) |
| Syria | Arabic (official); Kurdish, Armenian, Aramaic, Circassian |
| | widely understood; French, English somewhat understood |
| Taiwan | Chinese (Mandarin, official), Taiwanese (Min), Hakka |
| | dialects |
| Tajikistan | Tajik (official), Russian widely used in government and |
| | business |
| Tanzania | Swahili, English (both official); Arabic; many local |
| | languages |
| Thailand | Thai (Siamese), English (secondary language of the elite), |
| | ethnic and regional dialects |
| Togo | French (official, commerce); Ewé, Mina (south); Kabyé, |
| | Dagomba (north); and many dialects |
| Tonga | Tongan (an Austronesian language), English |
| Trinidad and | English (official), Hindi, French, Spanish, Chinese |
| Tobago | |
| Tunisia | Arabic (official, commerce), French (commerce) |
| Turkey | Turkish (official), Kurdish, Dimli, Azeri, Kabardian |
| Turkmenistan | Turkmen 72%; Russian 12%; Uzbek 9%, other 7% |
| Tuvalu | Tuvaluan, English, Samoan, Kiribati (on the island of Nui) |
| Uganda | English (official), Ganda or Luganda, other Niger-Congo |
| | languages, Nilo-Saharan languages, Swahili, Arabic |
| Ukraine | Ukrainian 67%, Russian 24%, Romanian, Polish, Hungarian |
| United Arab | Arabic (official), Persian, English, Hindi, Urdu |
| Emirates | |
| United | English, Welsh, Scots Gaelic |
| Kingdom | |
| United States | English 82%, Spanish 11% (2000) |
| Uruguay | Spanish, Portunol, or Brazilero |
| Uzbekistan | Uzbek 74.3%, Russian 14.2%, Tajik 4.4%, other 7.1% |
| Vanuatu | Bislama 23% (a Melanesian pidgin English), English 2%, |
| | French 1% (all 3 official); more than 100 local languages 73% |
| Vatican City | Italian, Latin, French, various other languages |
| (Holy See) | |
| Venezuela | Spanish (official), numerous indigenous dialects |
| Vietnam | Vietnamese (official); English (increasingly favored as a |
| | second language); some French, Chinese, Khmer; mountain |
| | area languages (Mon-Khmer and Malayo-Polynesian) |
| Western Sahara | Hassaniya Arabic, Moroccan Arabic |
| (proposed state) | |
| Yemen | Arabic |
| Zambia | English (official); major vernaculars: Bemba, Kaonda, Lozi, |
| | Lunda, Luvale, Nyanja, Tonga; about 70 other indigenous |
| | languages |
| Zimbabwe | English (official), Shona, Ndebele (Sindebele), numerous |
| | minor tribal dialects |

Category g:

Category g is used for identifying ROD's scale of scientific knowledge.

The category g database contents with different scientific knowledge are specifically causing remote operator's using devices of V2K & RNM ROD's slow down psychological response: lighten harassment or stop harassment, because the identification system administrator (ISA) let tested person TP intentionally imagine some knowledge of science or technology which ROD do not know.

The reason why ROD lighten harassment or stop harassment when they meet some knowledge of science and technology which they do not know, these ROD need time to understand and consider how response on their device of V2K & RNM, so these ROD lighten or stop their harassment to TP.

For examples, assume a TP has computer technology knowledge and know how change theses data among hexadecimal, binary and decimal. At this point, TP can change a hexadecimal data into a binary data, then change the binary data into decimal data within five seconds which are from database in computer.

We assume there are requirements in database of a computer asking the TP to answer the questions:

1. Please change B into binary, then change it into decimal.

The TP answer: binary is 1011, then decimal is 11 within five seconds.

2. Please change F into binary, then change it into decimal.

The TP answer: binary is 1111, then decimal is 15 within five seconds.

When these questions are processing, we can find an obvious phenomenon: TP feel or sense the remote harassment get weaken or even disappeared temporarily, the reason is that those ROD do not understand how change data among hexadecimal, binary and decimal, so those remote operators (ROD) have no way to response on the device of V2K & RNM as soon as possible.

All these ways can be repeatedly identified to confirm its accuracy.

After TP has chosen a kind of content from database g, he/she must follow instruction in a computer to launch a thoughts imagination psychological attack to ROD and then answer question in diamond frame in flowchart which shows on touch screen, then ISA checks PM to confirm TP's answer is true.

According automatic control theory, multiple TP's psychological attacking cycles with scientific knowledge will generate precise identification: the harassment become lighten or stops response comes from ROD, but these does not belong to TP's

FIG. 3A to FIG. 3E illustrate a second phase of flowchart.

Table 2 shows examples of different kinds of sciences.

TABLE 2
List of different kinds of sciences

| Name of Sciences | Description |
|---|---|
| Acoustics | The study of sound (or the science of sound) |
| Aerodynamics | The study of the motion and control of solid bodies |
| | like aircraft, missiles, etc., in air |
| Aeronautics | The science or art of flight |
| Aeronomy | The study of the earth's upper atmosphere, including |
| | its composition, density, temperature and chemical reactions, |
| | as recorded by sounding rockets and earth satellites |
| Aerostatics | The branch of statics that deals with gases in |
| | equilibrium and with gases and bodies in them |
| Aetiology | The science of causation |
| Agrobiology | The science of plant life and plant nutrition |
| Agronomy | The science of soil management and the production |
| | of field crops |
| Agrostology | The study of grasses |
| Alchemy | Chemistry in ancient times |
| Anatomy | The science dealing with the structure of animals, |
| | plants or human body |
| Anthropology | The science that deals with the origins, physical and |
| | cultural development of mankind |
| Arboriculture | Cultivation of trees and vegetables |
| Archaeology | The study of antiquities |
| Astrochemistry | The study of interstellar matter with a view to knowing |
| | the origin of universe |
| Astrology | The ancient art of predicting the course of human |
| | destinies with the help of indications deduced from the |
| | position and movement of the heavenly bodies |
| Astronautics | The science of space travel |
| Astronomy | The study of the heavenly bodies |
| Astrophysics | The branch of astronomy concerned with the physical |
| | nature of heavenly bodies |
| Autoecology | The study deals with the ecology of species |
| Bacteriology | The study of bacteria |
| Biochemistry | The study of chemical processes of living things |
| Bioclimatology | Studies the effects of climate upon living organisms |
| Biology | The study of living things |
| Biometry | The application of mathematics to the study of living |
| | things |
| Biomechanics | The study of the mechanical laws relating to the |
| | movement or structure of living organisms |
| Biometeorology | Studies the effects of atmospheric conditions on living |
| | organisms |
| Bionics | The study of functions, characteristics and phenomena |
| | observed in the living world and the application of this |
| | knowledge to the world of machines |
| Bionomics | The study of the relation of an organism to its |
| | environments |
| Bionomy | The science of the laws of life |
| Biophysics | The physics of vital processes (living things) |
| Botany | The study of plants |
| Ceramics | The art and technology of making objects from clay, |
| | etc. (pottery) |
| Chemistry | The study of elements and their laws of combination |
| | and behaviour |
| Chemotherpy | The treatment of disease by using chemical substances |
| Chronobiology | The study of the duration of life |
| Chronology | The science of arranging time in periods and |
| | ascertaining the dates and historical order of past |
| | events |
| Climatotherapy | The treatment of disease through suitable climatic |
| | environment, often, but not always, found in |
| | recognised health resorts. As climate is subject to |
| | seasonal variations, the required environment may |
| | have to be sought in different localities at different |
| | periods of the year |
| Conchology | The branch of zoology dealing with the shells of |
| | mollusks |
| Cosmogony | The science of the nature of heavenly bodies |
| Cosmography | The science that describes and maps the main features |
| | of the universe |
| Cryobiology | The science that deals with the study of organisms, |
| | especially warmblooded animals, at low temperature. |
| | The principal effect of cold on living tissues is |
| | destruction of life or preservation of it at a reduced |
| | level of activity |
| Crystallography | The study of the structure, forms and properties of |
| | crystals |
| Cryogenics | The science dealing with the production, control and |
| | application of very low temperatures |
| Cryotherapy | Use of cold, but not freezing cold, as a form of |
| | treatment. Hypothermia may be deliberately induced |
| | during surgery, for instance, to decrease a patient's |
| | oxygen requirement |
| Cytochemistry | The branch of cytology dealing with the chemistry of |
| | cells |
| Cytogenetics | The branch of biology dealing with the study of |
| | heredity from the point of view of cytology and |
| | genetics |
| Cytology | The study of cells, especially their formation, structure |
| | and functions |
| Dactylography | The study of fingerprints for the purpose of |
| | identification |
| Dermatology | The study of skin and skin diseases |
| Ecology | The study of the relation of animals and plants to their |
| | surroundings, animate and inanimate |
| Econometrics | The application of mathematics in testing economic |
| | theories |
| Economics | The science dealing with the production, distribution |
| | and consumption of goods and services |
| Electronics | Studies the development, behaviour and applications |
| | of electronic devices and circuits |
| Electrostatics | It is a study of static electricity |
| Embryology | The study of development of embryos |
| Entomology | The study of insects |
| Epidemiology | The branch of medicine dealing with epidemic |
| | diseases |
| Epigraphy | The study of inscriptions |
| Ethnography | A branch of anthropology dealing with the scientific |
| | description of individual cultures |
| Ethnology | A branch of anthropology that deals with the origin, |
| | distribution and distinguishing characteristics of the |
| | races of mankind |
| Ethology | The study of animal behaviour |
| Eugenics | The study of the production of better offspring by the |
| | careful selection of parents |
| Fractography | A study of fractures in metal surfaces |
| Genealogy | The study of family origins and history. It includes the |
| | compilation of lists of ancestors and arranging them in |
| | pedigree charts |
| Genecology | The study of genetical composition of plant population |
| | in relation to their habitats |
| Genesiology | The science of generation |
| Genetics | The branch of biology dealing with the phenomena of |
| | heredity and the laws governing it |
| Geobiology | The biology of terrestrial life |
| Geobotany | The branch of botany dealing with all aspects of |
| | relations between plants and the earth's surface |
| Geochemistry | The study of the chemical composition of the earth's |
| | crust and the changes which take place within it |
| Geodesy | Methods of surveying the earth for making maps and |
| | correlating geological, gravitational and magnetic |
| | measurements. It is a branch of geo-physics |
| Geography | The development of science of the earth's surface, physical |
| | features, climate, population, etc |
| Geology | The science that deals with the physical history of the |
| | earth |
| Geomedicine | The branch of medicine dealing with the influence of |
| | climate and environmental conditions on health |
| Geomorphology | The study of the characteristics, origin and |
| | development of land forms |
| Geophysics | The physics of the earth |
| Gerontology | The study of old age, its phenomena, diseases, etc |
| Glaciology | The study of ice and the action of ice in all its forms, |
| | and therefore including now |
| Gynaecology | A study of diseases of women's reproductive organs |
| Histology | The study of tissues |
| Horticulture | The cultivation of flowers, fruits, vegetables and |
| | ornamental plants |
| Hydrodynamics | The mathematical study of the forces, energy and |
| | pressure of liquid in motion |
| Hydrography | The science of water measurements of the earth with |
| | special reference to their use for navigation |
| Hydrology | The study of water with reference to its occurrence and |
| | properties in the hydrosphere and atmosphere |
| Hydrometallurgy | The process of extracting metals at ordinary |
| | temperature by bleaching ore with liquids |
| Hydrometeorology | The study of the occurrence, movement and changes in |
| | the state of water in the atmosphere |
| Hydropathy | The treatment of disease by the internal and external |
| | use of water |
| Hydroponics | The cultivation of plants by placing the roots in liquid |
| | nutrient solutions rather than in soil |
| Hydrostatics | The mathematical study of forces and pressures in liquids |
| Hygiene | The science of health and its preservation |
| Limnology | The study of lakes |
| Lithology | It deals with systematic description of rocks |
| Mammography | Radiography of the mammary glands |
| Metallography | The study of the crystalline structures of metals and |
| | alloys |
| Metallurgy | The process of extracting metals from their ores |
| Meteorology | The science of the atmosphere and its phenomena |
| Metrology | The scientific study of weights and measures |
| Microbiology | The study of minute living organisms, including |
| | bacteria, molds and pathogenic protozoa |
| Molecular biology | The study of the structure of the molecules which are |
| | of importance in biology |
| Morphology | The science of organic forms and structures |
| Mycology | The study of fungi and fungus diseases |
| Neurology | The study of the nervous system, its functions and its |
| | disorders |
| Neuropathology | The study of diseases of the nervous system |
| Nosology | The classification of diseases |
| Numerology | The study of numbers. The study of the date and year |
| | of one's birth and to determine the influence on one's |
| | future life |
| Odontology study | The scientific |
| of the teeth | |
| Optics | The study of nature and properties of light |
| Ornithology | The study of birds |
| Orthopedics | The science of prevention, diagnosis and treatment of |
| | diseases and abnormalities of musculoskeletal system |
| Osteology | The study of the bones |
| Osteopathy | A therapeutic system based upon detecting and |
| | correcting faulty structure |
| Otology | The study of the ear and its diseases |
| Otorhinolaryngology | Study of diseases of ear, nose and throat |
| Paleobotany | The study of fossil plants |
| Paleontology | The study of fossils |
| Pathology | The study of diseases |
| Pharyngology | The science of the pharynx and its diseases |
| Phenology | The study of periodicity phenomena of plants |
| Philology | The study of written records, their authenticity, etc |
| Phonetics | The study of speech sounds and the production, |
| | transmission, reception, etc |
| Photobiology | The branch of biology dealing with the effect of light |
| | on organisms |
| Phrenology | The study of the faculties and qualities of minds from |
| | the shape of the skull |
| Phthisiology | The scientific study of tuberculosis |
| Phycology | The study of algae |
| Physical Science | The study of natural laws and processes other than |
| | those peculiar to living matters, as in physics, |
| | chemistry and astronomy |
| Physics | The study of the properties of matter |
| Physiography | The science of physical geography |
| Physiology | The study of the functioning of the various organs of |
| | living beings |
| Phytogeny | The science dealing with origin and growth of plants |
| Planetology | A study of the planets of the Solar System |
| Pomology | The science that deals with fruits and fruit growing |
| Psychology | The study of human and animal behaviour |
| Radio Astronomy | The study of heavenly bodies by the reception and |
| | analysis of the radio frequency electro-magnetic |
| | radiations which they emit or reflect |
| Radiobiology | The branch of biology which deals with the effects of |
| | radiations on living organisms |
| Radiology | The study of X-rays and radioactivity |
| Rheology | The study of the deformation and flow of matter |
| Seismology | The study of earthquakes and the phenomena |
| | associated with it |
| Selenology | The scientific study of moon, its nature, origin, |
| | movement, etc |
| Sericulture | The raising of silkworms for the production of raw silk |
| Sociology | The study of human society |
| Spectroscopy | The study of matter and energy by the use of |
| | spectroscope |
| Statistics | The collection and analysis of numerical data |
| Tectonics | Study of structural features of earth's crust |
| Teleology | The study of the evidences of design or purpose in |
| | nature |
| Telepathy | Communication between minds by some means other |
| | than sensory perception |
| Therapeutics | The science and art of healing |
| Topography | A special description of a part or region |
| Toxicology | The study of poisons |
| Virology | The study of viruses |
| Zoogeography | The study of the geological distributions of animals |
| Zoology | The study of animal life |

All these identifies can be repeatedly done to confirm the last identification is correct, there is not contingency.

The operating method of the present invention can refer to FIG. 1, FIG. 2A to FIG. 2F, FIG. 10, FIG. 11A, FIG. 11B, FIG. 12A, FIG. 12B, FIG. 13, FIG. 14, and FIG. 3A to FIG. 3E of the drawings.

FIG. 4 can explain why the reason the method of identification of the present invention can get accurate result.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

1: An improved method to identify a victim of abuse and a remote operator using a device of voice to skull and remote neural monitoring, comprising the steps of:
selecting one item from a database (a) in a computer by a tested person which only causes the remote operator to have a disgusting psychological response but does not cause the tested person to have the disgusting psychological response, instructing the tested person to launch a first imaginary psychological attack, assigning a first set of test questions based on the first imaginary psychological attack and obtaining a first set of answers from the tested person in response to the first set of test questions by an automatic system with a human machine interface communicating to a controller, and analyzing the first set of answers and assigning a true or false status to the first set of answers by a polygraph meter connecting to the controller;
selecting one item from a database (b) in the computer which only causes the remote operator to have a funny psychological response but does not cause the tested person to have the funny psychological response, instructing the tested person to launch a second imaginary psychological attack, assigning a second set of test questions based on the second imaginary psychological attack and obtaining a second set of answers from the tested person in response to the second set of test questions by the automatic system with the human machine interface communicating to the controller, and analyzing the second set of answers and assigning a true or false status to the second set of answers by the polygraph meter; and
using a database (e) in the computer consisting of a plurality of foreign clubs or organizations items, the tested person selecting one of the foreign clubs or organizations items which only causes the remote operator to have a foreign clubs or organizations psychological response but does not cause the tested person to have the foreign clubs or organizations psychological response, instructing the tested person to launch a third imaginary psychological attack, assigning a third set of test questions based on the third imaginary psychological attack and obtaining a third set of answers from the tested person in response to the third set of test questions by the automatic system with the human machine interface communicating to the controller, and analyzing the third set of answers by the polygraph meter and assigning a true or false status to the third set of answers, where the foreign clubs or organizations psychological response is angry psychological response and strong deny.

2: The improved method to identify the victim of abuse and the remote operator using the device of voice to skull and remote neural monitoring according to claim 1, further comprising the steps of:
selecting one item from a database (c) in the computer which only causes the remote operator to have an anti-political trend and anti-religious belief psychological response and does not cause the tested person to have the anti-political trend and anti-religious belief psychological response, instructing the tested person to launch a fourth imaginary psychological attack, assigning a fourth set of test questions based on the fourth imaginary psychological attack and obtaining a fourth set of answers from the tested person in response to the fourth set of test questions, the and analyzing the fourth set of answers by the polygraph meter and assigning a true or false status to the fourth set of answers.

3: The improved method to identify the victim of abuse and the remote operator using the device of voice to skull and remote neural monitoring according to claim 1, further comprising the steps of:
using a database (d) in the computer consisting of a plurality of countries and races items, the tested person selecting one of the countries and races items which only causes the remote operator to have a countries and races psychological response and does not cause the tested person to have the countries and races psychological response, instructing the tested person to launch a fifth imaginary psychological attack, assigning a fifth set of test questions based on the fifth imaginary psychological attack and obtaining a fifth set of answers from the tested person in response to the fifth set of test questions, and analyzing the fifth set of answers by the polygraph meter and assigning a true or false status to the fifth set of answers.

4: The improved method to identify the victim of abuse and the remote operator using the device of voice to skull and remote neural monitoring according to claim 2, further comprising the steps of:
using a database (d) in the computer consisting of a plurality of countries and races items, the tested person selecting one of the countries and races items which only causes the remote operator to have a countries and races psychological response and does not cause the tested person to have the countries and races psychological response, instructing the tested person to launch a fifth imaginary psychological attack, assigning a fifth set of test questions based on the fifth imaginary psychological attack and obtaining a fifth set of answers from the tested person in response to the fifth set of test questions, and analyzing the fifth set of answers by the polygraph meter and assigning a true or false status to the fifth set of answers.

5: The improved method to identify the victim of abuse and the remote operator using the device of voice to skull and remote neural monitoring according to claim 1, further comprising the steps of:
using a database (f) in the computer consisting of a plurality of languages and dialects items, the tested person selecting one of the languages and dialects items which only causes the remote operator to have a languages and dialects psychological response and does not cause the tested person to have the languages and dialects psychological response, instructing the tested person to launch a sixth imaginary psychological attack, assigning a sixth set of test questions based on the sixth imaginary psychological attack and obtaining a sixth set of answers from the tested person in response to the sixth set of test questions, and analyzing the sixth set of answers by the polygraph meter and assigning a true or false status to the sixth set of answers.

6: The improved method to identify the victim of abuse and the remote operator using the device of voice to skull and remote neural monitoring according to claim 2, further comprising the steps of:
using a database (f) in the computer consisting of a plurality of languages and dialects items, the tested person selecting one of the languages and dialects items which only causes the remote operator to have a languages and dialects psychological response and does not cause the tested person to have the languages and dialects psychological response, instructing the tested person to launch a sixth imaginary psychological attack, assigning a sixth set of test questions based on the sixth imaginary psychological attack and obtaining a sixth set of answers from the tested person in response to the sixth set of test questions, and analyzing the sixth set of answers by the polygraph meter and assigning a true or false status to the sixth set of answers.

7: The improved method to identify the victim of abuse and the remote operator using the device of voice to skull and remote neural monitoring according to claim 3, further comprising the steps of:
using a database (f) in the computer consisting of a plurality of languages and dialects items, the tested person selecting one of the languages and dialects items which only causes the remote operator to have a languages and dialects psychological response and does not cause the tested person to have the languages and dialects psychological response, instructing the tested person to launch a sixth imaginary psychological attack, assigning a sixth set of test questions based on the sixth imaginary psychological attack and obtaining a sixth set of answers from the tested person in response to the sixth set of test questions, and analyzing the sixth set of answers by the polygraph meter and assigning a true or false status to the sixth set of answers.

8: The improved method to identify the victim of abuse and the remote operator using the device of voice to skull and remote neural monitoring according to claim 4, further comprising the steps of:
using a database (f) in the computer consisting of a plurality of languages and dialects items, the tested person selecting one of the languages and dialects items which only causes the remote operator to have a languages and dialects psychological response and does not cause the tested person to have the languages and dialects psychological response, instructing the tested person to launch a sixth imaginary psychological attack, assigning a sixth set of test questions based on the sixth imaginary psychological attack and obtaining a sixth set of answers from the tested person in response to the sixth set of test questions, and analyzing the sixth set of answers by the polygraph meter and assigning a true or false status to the sixth set of answers.

9: The improved method to identify the victim of abuse and the remote operator using the device of voice to skull and remote neural monitoring according to claim 1, further comprising the steps of:
using a database (g) in the computer consisting of a plurality of scientific knowledge items, the tested person selecting one of the scientific knowledge items which only causes the remote operator to have a scientific knowledge response and does not cause the tested person to have the scientific knowledge response, instructing the tested person to launch a seventh imaginary psychological attack, assigning a seventh set of test questions based on the seventh imaginary psychological attack and obtaining a seventh set of answers from the tested person in response to the seventh set of test questions, then using polygraph meter to analysis the seventh set of answers and assigning a true or false status to the seventh set of answers.

10: The improved method to identify the victim of abuse and the remote operator using the voice to skull remote neural monitoring according to claim 2, further comprising the steps of:
using a database (g) in the computer consisting of a plurality of scientific knowledge items, the tested person selecting one of the scientific knowledge items which only causes the remote operator to have a scientific knowledge response and does not cause the tested person to have the scientific knowledge response, instructing the tested person to launch a seventh imaginary psychological attack, assigning a seventh set of test questions based on the seventh imaginary psychological attack and obtaining a seventh set of answers from the tested person in response to the seventh set of test questions, then using polygraph meter to analysis the seventh set of answers and assigning a true or false status to the seventh set of answers.

11: The improved method to identify the victim of abuse and the remote operator using the device of voice to skull and remote neural monitoring according to claim 3, further comprising the steps of:
using a database (g) in the computer consisting of a plurality of scientific knowledge items, the tested person selecting one of the scientific knowledge items which only causes the remote operator to have a scientific knowledge response and does not cause the tested person to have the scientific knowledge response, instructing the tested person to launch a seventh imaginary psychological attack, assigning a seventh set of test questions based on the seventh imaginary psychological attack and obtaining a seventh set of answers from the tested person in response to the seventh set of test questions, then using polygraph meter to analysis the seventh set of answers and assigning a true or false status to the seventh set of answers.

12: The improved method to identify the victim of abuse and the remote operator using the device of voice to skull and remote neural monitoring according to claim 4, further comprising the steps of:
using a database (g) in the computer consisting of a plurality of scientific knowledge items, the tested person selecting one of the scientific knowledge items which only causes the remote operator to have a scientific knowledge response and does not cause the tested person to have the scientific knowledge response, instructing the tested person to launch a seventh imaginary psychological attack, assigning a seventh set of test questions based on the seventh imaginary psychological attack and obtaining a seventh set of answers from the tested person in response to the seventh set of test questions, then using polygraph meter to analysis the seventh set of answers and assigning a true or false status to the seventh set of answers.

13: The improved method to identify the victim of abuse and the remote operator using the device of voice to skull and remote neural monitoring according to claim 5, further comprising the steps of:
using a database (g) in the computer consisting of a plurality of scientific knowledge items, the tested person selecting one of the scientific knowledge items which only causes the remote operator to have a scientific knowledge response and does not cause the tested person to have the scientific knowledge response, instructing the tested person to launch a seventh imaginary psychological attack, assigning a seventh set of test questions based on the seventh imaginary psychological attack and obtaining a seventh set of answers from the tested person in response to the seventh set of test questions, then using polygraph meter to analysis the seventh set of answers and assigning a true or false status to the seventh set of answers.

14: The improved method to identify the victim of abuse and the remote operator using the device of voice to skull and remote neural monitoring according to claim 6, further comprising the steps of:
using a database (g) in the computer consisting of a plurality of scientific knowledge items, the tested person selecting one of the scientific knowledge items which only causes the remote operator to have a scientific knowledge response and does not cause the tested person to have the scientific knowledge response, instructing the tested person to launch a seventh imaginary psychological attack, assigning a seventh set of test questions based on the seventh imaginary psychological attack and obtaining a seventh set of answers from the tested person in response to the seventh set of test questions, then using polygraph meter to analysis the seventh set of answers and assigning a true or false status to the seventh set of answers.

15-16. (canceled)

17: The improved method to identify the victim of abuse and the remote operator using the device of voice to skull and remote neural monitoring according to claim 8, further comprising the steps of:
using a database (g) in the computer consisting of a plurality of scientific knowledge items, the tested person selecting one of the scientific knowledge items which only causes the remote operator to have a scientific knowledge response and does not cause the tested person to have the scientific knowledge response, instructing the tested person to launch a seventh imaginary psychological attack, assigning a seventh set of test questions based on the seventh imaginary psychological attack and obtaining a seventh set of answers from the tested person in response to the seventh set of test questions, then using polygraph meter to analysis the seventh set of answers and assigning a true or false status to the seventh set of answers.

18-20. (canceled)
